(19) United States
(12) Patent Application Publication
(10) Pub. No.: US 2021/0052589 A1
(43) Pub. Date: Feb. 25, 2021

(54) COMPOUNDS FOR TREATING ALZHEIMER'S DISEASE

(71) Applicant: Amabiotics, Paris (FR)

(72) Inventors: Patrice Garnier, Venezia (IT); Antoine Danchin, Paris (FR)

(73) Assignee: Amabiotics, Paris (FR)

(21) Appl. No.: 16/977,979

(22) PCT Filed: Mar. 7, 2019
(86) PCT No.: PCT/EP2019/055780

§ 371 Date: Sep. 3, 2020

(30) Foreign Application Priority Data
Mar. 7, 2018 (EP) 18305245.5

(51) Int. Cl. A61K 31/5025; A61K 9/00; A23L 29/00

(52) U.S. Cl. CPC A61K 31/5025; A61K 9/0019; A61K 31/44; A23L 29/045; A61K 9/0053

(57) ABSTRACT

The present invention relates to a compound of the following formula (I): (I) or a pharmaceutically acceptable salt or hydrate thereof, for use in the prevention or treatment of Alzheimer's disease in an individual.

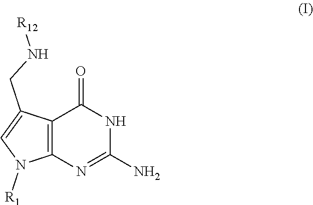

### FIELD OF THE INVENTION

The present invention relates to compounds, compositions and methods for treating Alzheimer's disease.

### BACKGROUND OF THE INVENTION

Alzheimer's disease is the most common neurodegenerative disease and the most common cause of dementia in the elderly. By September 2015, the disease affected 47.5 million people worldwide. Its prevalence is 5% after 65 years old and grows exponentially with age (25% of people aged more than 80 are affected) but it can also occur much earlier.

Alzheimer's disease is characterised by progressive and irreversible loss of neurons. This loss leads to the decline of cognitive faculties, such as memory, language, reasoning, as well as a disappearance of orientation capacities in time and space. Eventually, patients are unable to recognise familiar people or to carry out mundane tasks.

Alzheimer's disease appears to be due, at least in part, to the presence in the brain of extracellular deposits of β-amyloid peptide forming so-called amyloid plaques. These plaques lead to a dysfunction of surrounding neurons, and ultimately to neuronal death. The β-amyloid peptide comprises 36 to 43 amino acids and is derived from an abnormal enzymatic cleavage of the amyloid precursor protein (APP), by β-secretase and γ-secretase. This peptide is insoluble and little degraded. This process usually starts at the hippocampus and gradually extends to different areas of the cerebral cortex.

At this time, there is no drug treatment for this disease.

Indeed, current drugs approved in this context, such as donepezil (Rogers et al. (1998) Arch Intern Med. 158: 1021-3), an anti-cholinesterase, aim at slowing the symptomatic progression of the disease, but do not prevent the neuronal degeneration and death.

Accordingly, there is a need for alternative treatments to these compounds in Alzheimer's disease, in particular to alter the pathological mechanisms of this condition in addition to treating the symptoms.

### SUMMARY OF THE INVENTION

The present invention arises from the recognition, by the present inventors, of the potential of queuine as a neuroprotective agent in the prevention or treatment of Alzheimer's disease. Without wishing to be bound to a particular theory, the inventors believe that queuine could prevent or exert a beneficial action on the misfolding of proteins involved in the pathological mechanism leading to amyloid plaque formation.

Thus, the present invention relates to queuine, a precursor of queuine, a derivative of queuine or a stereoisomer of queuine, an analogue of queuine, or a pharmaceutically acceptable salt or hydrate thereof, for use in the prevention or treatment of Alzheimer's disease.

The present invention relates to a compound of the following formula (I):

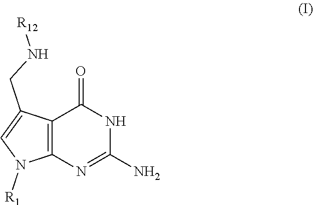

wherein:
R₁ represents —H or a ribosyl group of the following formula:

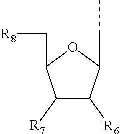

wherein:
   R₆ represents —H; —O—R₉ or —O—CO—R₉ wherein R₉ is H, an alkyl group having from 1 to 6 carbon atoms or an aryl group having from 3 to 12 carbon atoms;
   R₇ represents —H; —O—R₁₀ or —O—CO—R₁₀ wherein R₁₀ is H, an alkyl group having from 1 to 6 carbon atoms or an aryl group having from 3 to 12 carbon atoms; a deoxyribonucleic acid group; or a ribonucleic acid group;
   R₈ represents —H; —O—R₁₁ or —O—CO—R₁₁ wherein R₁₁ is H, an alkyl group having from 1 to 20 carbon atoms or an aryl group having from 3 to 20 carbon atoms; a phosphate group; a diphosphate group; a triphosphate group; a deoxyribonucleic acid group; or a ribonucleic acid group;
R₁₂ represents a saturated or unsaturated alkyl, cycloalkyl, heterocycloalkyl or ether group having from 1 to 20 carbon atoms, optionally substituted by at least one group selected from the group consisting of:
   an alkyl group having from 1 to 20 carbon atoms,
   an aryl or heteroaryl group having from 3 to 20 carbon atoms,
   a cycloalkyl or heterocycloalkyl group having from 3 to 20 carbon atoms,
   a hydroxyl group,
   a carbonyl or carboxyl group having from 1 to 20 carbon atoms,
   an epoxy group,
   an —O—R₄ group wherein R₄ is H, an alkyl group having from 1 to 6 carbon atoms, an aryl group having from 3 to 12 carbon atoms, a glycosyl group or an aminoacyl group,
   an —O—CO—R₅ group wherein R₅ is an alkyl group having from 1 to 6 carbon atoms, an aryl group having from 3 to 12 carbon atoms or a glycosyl group;
      or a pharmaceutically acceptable salt or hydrate thereof,
      for use in the prevention or treatment of Alzheimer's disease in an individual.

The present invention also relates to a compound of the following formula (I) of the following formula (II):

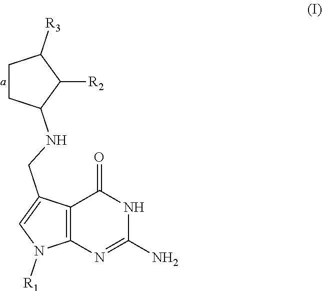

wherein:
a represents a double bond or an epoxy group, and
R₁ represents —H or a ribosyl group of the following formula:

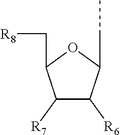

wherein:
   R₆ represents —H; —O—R₉ or —O—CO—R₉ wherein R₉ is H, an alkyl group having from 1 to 6 carbon atoms or an aryl group having from 3 to 12 carbon atoms;
   R₇ represents —H; —O—R₁₀ or —O—CO—R₁₀ wherein R₁₀ is H, an alkyl group having from 1 to 6 carbon atoms or an aryl group having from 3 to 12 carbon atoms; a deoxyribonucleic acid group; or a ribonucleic acid group;
   R₈ represents —H; —O—R₁₁ or —O—CO—R₁₁ wherein R₁₁ is H, an alkyl group having from 1 to 20 carbon atoms or an aryl group having from 3 to 20 carbon atoms; a phosphate group; a diphosphate group; a triphosphate group; a deoxyribonucleic acid group; or a ribonucleic acid group;
R₂ and R₃, which are identical or different, represent —O—R₄ wherein R₄ is H, an alkyl group having from 1 to 6 carbon atoms, an aryl group having from 3 to 12 carbon atoms, a glycosyl group or an aminoacyl group; or —O—CO—R₅ wherein R₅ is an alkyl group having from 1 to 6 carbon atoms, an aryl group having from 3 to 12 carbon atoms or a glycosyl group;
   or a pharmaceutically acceptable salt or hydrate thereof,
   for use in the prevention or treatment of Alzheimer's disease in an individual.

In an embodiment of the invention the compound of formula (I), in particular the compound of formula (II), or the pharmaceutically acceptable salt or hydrate thereof, for use as defined above, is in combination with at least one additional compound useful for the prevention or treatment of Alzheimer's disease.

The present invention also relates to a pharmaceutical composition comprising as active substance a compound of formula (I), in particular a compound of formula (II), or a pharmaceutically acceptable salt or hydrate thereof as defined above, optionally in association with at least one pharmaceutically acceptable excipient or vehicle, for use in the prevention or treatment of Alzheimer's disease.

In an embodiment of the present invention the above defined pharmaceutical composition further comprises at least one additional compound useful for the prevention or treatment of Alzheimer's disease.

The present invention also relates to a pharmaceutical composition comprising as active substance a compound of formula (I), in particular a compound of formula (II), or a pharmaceutically acceptable salt or hydrate thereof, as defined above, further comprising at least one additional compound useful for the prevention or treatment of Alzheimer's disease, optionally in association with at least one pharmaceutically acceptable excipient or vehicle.

The present invention also relates to products comprising:

a compound of formula (I), in particular a compound of formula (II), or a pharmaceutically acceptable salt or hydrate thereof, as defined above,

at least one additional compound useful for the prevention or treatment of Alzheimer's disease,

as a combined preparation for simultaneous, separated or sequential use in the prevention or treatment of Alzheimer's disease in an individual.

The present invention also relates to a dietary supplement comprising a compound of formula (I), in particular a compound of formula (II), or a pharmaceutically acceptable salt or hydrate thereof, as defined above, for use for reducing the risk of Alzheimer's disease.

In an embodiment of the present invention, the dietary supplement as defined above, optionally comprises additional compounds, preferably selected from the group consisting of vitamins, minerals, fatty acids, amino acids and antioxidants.

The present invention also relates to a method for the prevention or treatment of Alzheimer's disease in an individual, comprising administering to the individual an effective amount of a compound of formula (I), in particular of a compound of formula (II), or of a pharmaceutically acceptable salt or hydrate thereof, as defined above.

In an embodiment of the present invention, the method as defined above further comprises the administration of at least one compound useful for the prevention or treatment of Alzheimer's disease.

The present invention also relates to the use of a compound of formula (I), in particular a compound of formula (II), as defined above for the manufacture of a medicament intended for the prevention or treatment of Alzheimer's disease in an individual.

In an embodiment of the present invention, the medicament as defined above further comprises at least one compound useful for the prevention or treatment of Alzheimer's disease.

### DETAILED DESCRIPTION OF THE INVENTION

### Compound of Formula (I)

Compounds of formula (I) as defined above can be readily chemically synthesized by one of skilled in the art, as is in particular described in Barnett & Grubb (2000), Tetrahedron 56: 9221-9225, Oxenford et al. (2004) Tetrahedron Letters 45:9053-9055, Brooks et al. (2010) Tetrahedron Letters 51: 4163-4165, Gerber et al. (2012) Org. Biomol. Chem. 10: 8660-8668, the thesis by Allen Brook entitled “Synthesis of Tritium Labeled Queuine, PreQ₁ and Related Azide Probes Toward Examining the Prevalence of Queuine” (2012, University of Michigan), Akimoto et al. (1986) J. Med. Chem., 29: 1749-1753, Kelly et al. (2016) Nucleic Acids Research, 1-11, and international application WO2016/050806, all of which are incorporated herein by reference.

Briefly, by way of example, queuine can be synthesised according to the following reaction scheme:

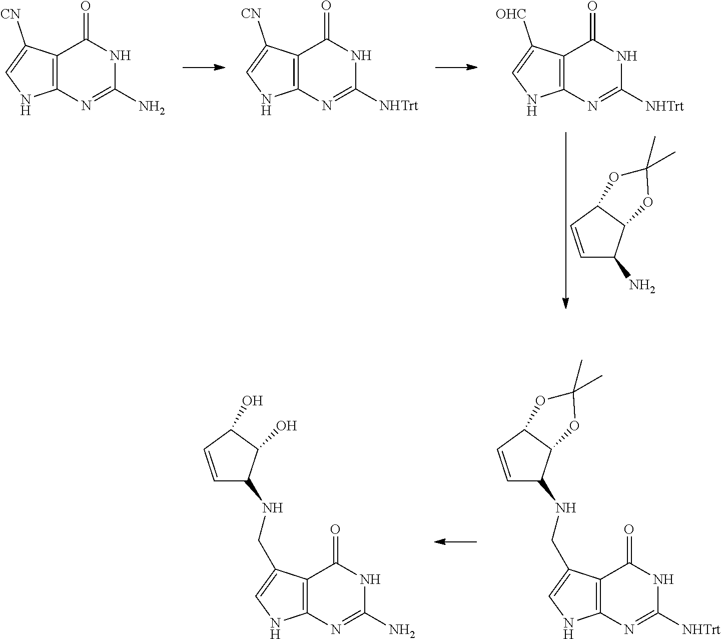

In additions, compounds of formula (I) as defined above may be extracted and optionally purified from natural sources such as microorganisms, in particular bacteria, or from plants, in particular from plants nodulated with alpha-Proteobacteria such as bacteria of the Rhizobium, Mesorhizobium, and Sinorhizobium genii.

By way of example, queuosine can be obtained from tRNAs, in particular tRNA^{Asn}, tRNA^{Asp}, tRNA^{His} and tRNA^{Tyr}, prepared as follows:

Preparation of Total RNA Under Acidic Conditions
B. subtilis strains (or other relevant bacteria) are grown in ED liquid medium with appropriate supplements at 37° C. with constant aeration. Fresh overnight cultures are inoculated in 15 ml of ED medium to an optical density at 600 nm (OD600) of 0.1. Cells are grown at 37° C. to OD600 of 1 and chilled in equal volume of 60% methanol in 70 mM Hepes pH 7.5 at −80° C. All subsequent steps are carried out in the cold and the solution for prepared crude RNA are treated with diethyl pyrocarbonate and sterilized. Cells are pelleted at 4° C., washed in water and re-suspended in 0.5 ml of 10% glucose, 11 mM Tris, 10 mM EDTA. The suspensions are transferred to tubes containing 0.1 g glass beads acid-washed (sigma-Aldrich, G4649). Tubes are disposed into The CoolPrep Adapter of FastPrep-24 Instrument (MP Biomedicals) containing 50 g of dry ice. Cells are broken after three cycles using following parameters: 6 meters per second during 45 s. After each cycle, suspensions are kept 1 min on ice. After centrifugation 2 min at 10,000 rpm, the supernatants are transferred to a fresh Eppendorf tube. 0.3 M sodium acetate pH 5.2 is added and total RNA is isolated under acidic conditions. One volume of acid phenol: chloroform with Isoamyl alcohol (125:24:1) pH 4.5 (Amresco, AM9720) is added. The sample are mixed by vortexing 10s and incubated for 3 min in a 65° C. water-bath. The phases are separated by spinning 5 min at 14,000 rpm, then the aqueous phase is re-extracted once with the same hot acid phenol procedure. The aqueous phase is transferred to a new tube and supplemented by one volume of cold acid phenol. After centrifugation 5 min at 14,000 rpm, RNA are precipitated with 2.5 volumes of absolute ethanol 1 h at −80° C. The RNA is pelleted at 14,000 rpm for 15 min at 4° C. and washed with 70% ethanol. The RNA pellet is dissolved in 10 mM Tris, 1 mM EDTA pH 7.5.

Enrichment of tRNA
The total RNA preparation is then mixed with one volume of lithium chloride 8 M pH 4.5 and sodium acetate pH 5.2 at 0.01 mM final concentration. This RNA solution is incubated 2 h at −80° C. After centrifugation at 14,000 rpm for 15 min at 4° C., the tRNA remained in supernatant. To remove salt contamination, tRNA is precipitated 1h at −80° C. by addition of 0.3 M sodium acetate pH 5.2 and 2.5 volumes of absolute ethanol. Then, tRNA is pelleted by centrifugation at 14,000 rpm for 15 min at 4° C. and washed with 70% ethanol. The tRNA pellet is dissolved in 10 mM Tris, 1 mM EDTA pH 7.5.

The stereoisomer of queuine according to the invention can be of any type. Preferably, the stereoisomer of queuine is ent-queuine.

The pharmaceutical acceptable salt or hydrate according to the invention can be of any type. However, it is preferred that the pharmaceutical acceptable salt according to the invention is a hydrochloride salt.

Preferably, the glycosyl group according to the invention is selected from the group consisting of a mannosyl group, a galactosyl group or a glutamyl group.

Preferably, the aminoacyl group is selected from alanine (ala, A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y) and valine (val, V).

In an embodiment of the invention, the substituents of formula (I), in particular of formula (II), according to the invention may be linked together.

In a preferred embodiment of the compound of formula (I) as defined above:

R₁ is H, and

R₁₂ represents a saturated or unsaturated alkyl, cycloalkyl, heterocycloalkyl or ether group having from 1 to 20 carbon atoms, optionally substituted by at least one group selected from the group consisting of:
an alkyl group having from 1 to 20 carbon atoms,
an aryl or heteroaryl group having from 3 to 20 carbon atoms,
a cycloalkyl or heterocycloalkyl group having from 3 to 20 carbon atoms,
a hydroxyl group,
a carbonyl or carboxyl group having from 1 to 20 carbon atoms,
an epoxy group,
an —O—R₄ group wherein R₄ is H, an alkyl group having from 1 to 6 carbon atoms, an aryl group having from 3 to 12 carbon atoms, a glycosyl group or an aminoacyl group,
an —O—CO—R₅ group wherein R₅ is an alkyl group having from 1 to 6 carbon atoms, an aryl group having from 3 to 12 carbon atoms or a glycosyl group.

In another preferred embodiment of the compound of formula (I) as defined above:

R₁₂ represent a group of the following formula:

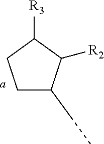

wherein:
a represents a double bond or an epoxy group, and
R₂ and R₃, which are identical or different, represent —O—R₄ wherein R₄ is H, an alkyl group having from 1 to 6 carbon atoms, an aryl group having from 3 to 12 carbon atoms, a glycosyl group or an aminoacyl group; or —O—CO—R₅ wherein R₅ is an alkyl group having from 1 to 6 carbon atoms, an aryl group having from 3 to 12 carbon atoms or a glycosyl group.

In another preferred embodiment of the compound of formula (I), as defined above:

R₁ is H, and

R₁₂ represents a saturated or unsaturated alkyl group having from 1 to 20 carbon atoms, optionally substituted by at least one of a hydroxyl group.

In another preferred embodiment of the compound of formula (I), in particular of the compound of formula (II), as defined above:

R₂ and R₃, which are identical or different, represent —OH, a —O-mannosyl group, a —O-galactosyl group or a —O-glutamyl group;

R₆ represents —OH;

R₇ and R₈, which are identical or different, represent —OH or a ribonucleic acid group.

Preferably, when R₇ and R₈ both represent a ribonucleic acid group, the compound of formula (I) according to the invention is included in a transfer RNA (tRNA) as a ribonucleoside of the tRNA. More preferably, the compound of formula (I) according to the invention is a ribonucleoside of the anticodon of the tRNA, most preferably the first nucleoside of the anticodon, i.e. the 5′ nucleoside of the anticodon or the nucleoside in the wobble position of the anticodon. Preferred tRNAs according to the invention are selected from the list consisting of tRNAs, tRNA^{Asn}, tRNA^{His} and tRNA^{Tyr}.

Preferably, the compound of formula (I), in particular the compound of formula (II), as defined above is represented by the following formulae (III), (IV) or (V):

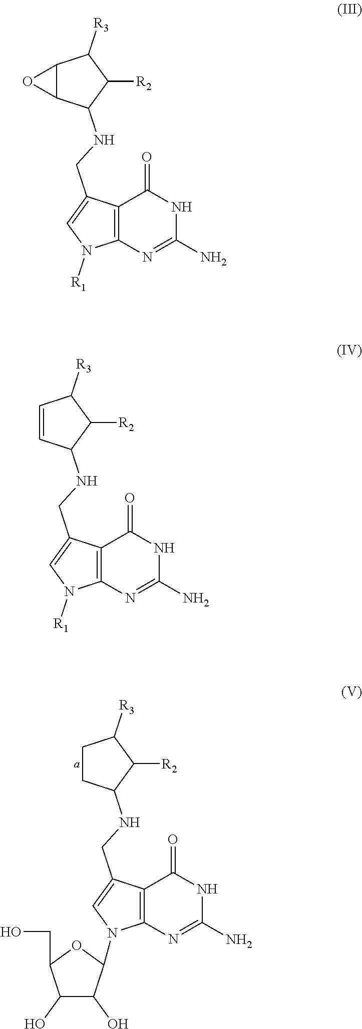

Preferably, when a compound of formula (I), in particular a compound of formulae (III)-(V) according to the invention is included in a tRNA^{Asp}, then R₃ is OH and R₂ is O-mannose.

Preferably also, when a compound of formula (I), in particular a compound of formulae (III)-(V) according to the invention is included in a tRNA^{Tyr}, then R₃ is OH and R₂ is O-galactose.

Preferably, the compound of formula (I), in particular the compound of formula (II) according to the invention is represented by the following formula (VI):

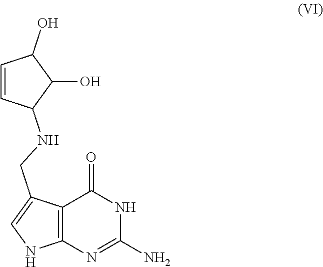 [SMILES: Nc1nc2[nH]cc(CNC3C=CC(O)C3O)c2c(=O)[nH]1]

As should be clear to one of skilled in the art, all the stereochemical configurations of the compounds according to the invention are intended to be covered by the formulae shown herein. In particular, as is intended herein, when the stereoconfiguration of a bond is not specified, the bond may represent any of an upward bond, a downward bond, and a mixture of the two, in particular a 1/1 mixture of the two.

Thus, the compound of formula (I) according to the invention also relates to the optically active forms of the compound of formula (V), such as the enantiomers represented by the following formulae (Va) and (Vb):

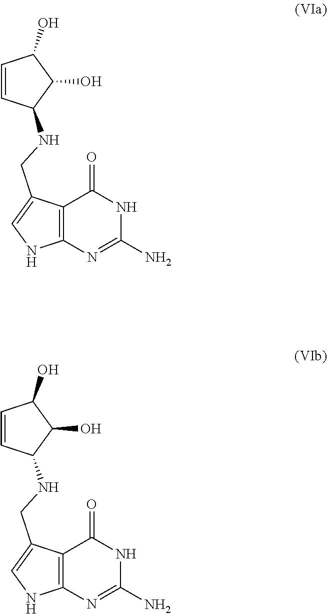 [SMILES: Nc1nc2[nH]cc(CN[C@H]3C=C[C@H](O)[C@@H]3O)c2c(=O)[nH]1 ; Nc1nc2[nH]cc(CN[C@@H]3C=C[C@@H](O)[C@H]3O)c2c(=O)[nH]1]

or their mixtures, in particular a racemic mixture thereof.

The compound of formula (VIa) is queuine. Queuine, is also known as 7-(3,4-trans-4,5-cis-dihydroxy-1-cyclopenten-3-ylaminomethyl)-7-deazaguanine. The compound of formula (VIb) is ent-queuine.

Preferably, the compound of formula (I), notably the compound of formula (II) according to the invention is represented by the following formulae (VII), (VIIa), (VIIb), (VIII), (VIIa), (VIIIb), (IX), (IXa) (IXb), (X)

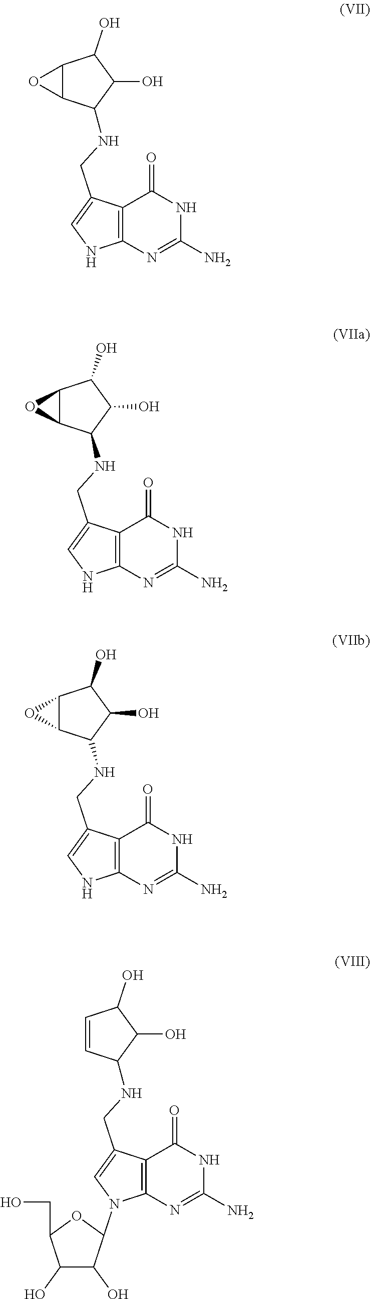

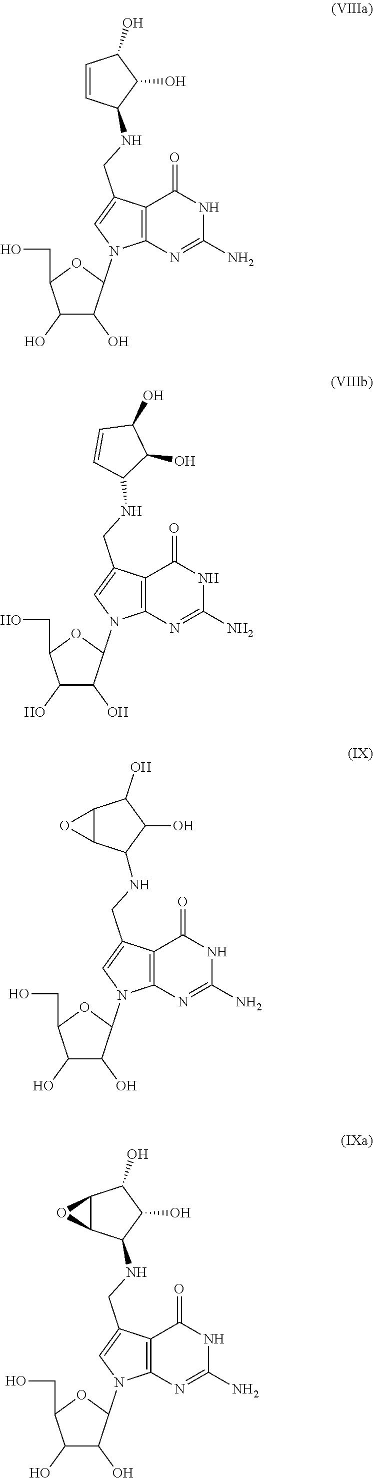

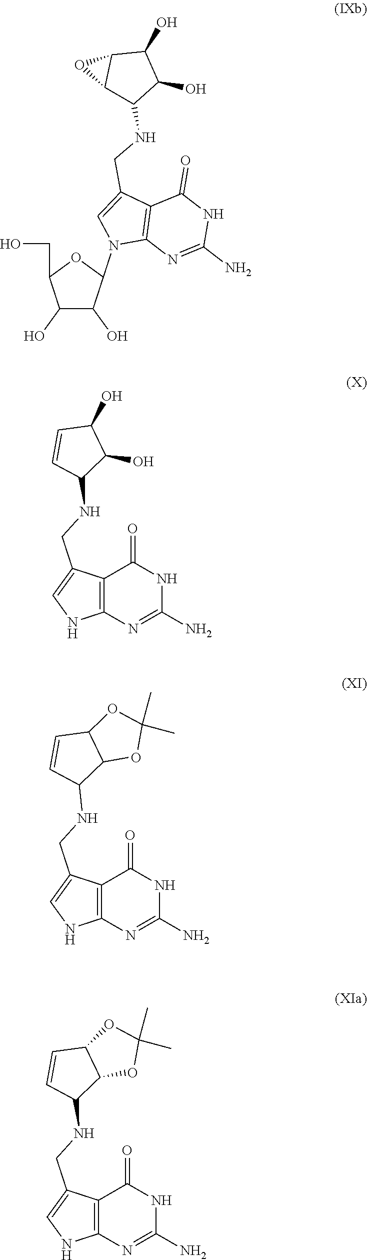

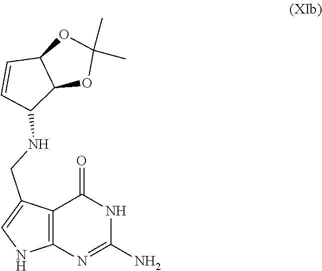

Preferably also, the compound of formula (I) according to the invention is represented by the following formulae (XII), (XIIa), (XIIb), (XIII), (XIV), (XV), (XVI), (XVII) or(XVIII):

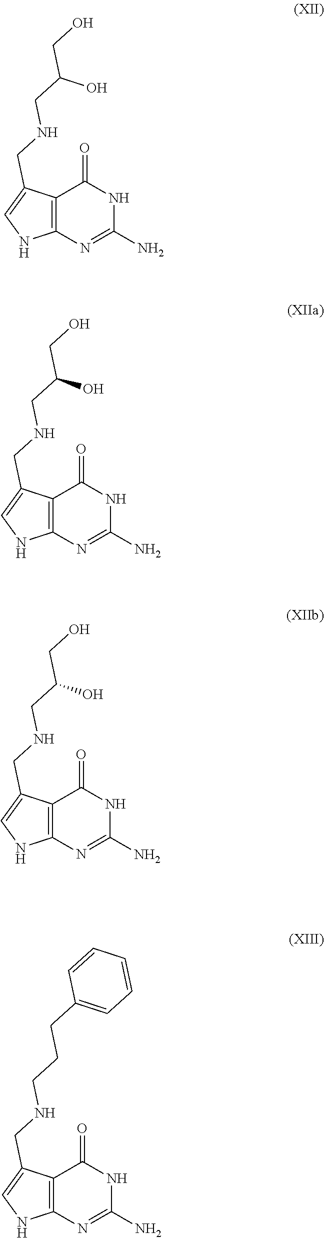 [SMILES: Nc1nc2[nH]cc(CNCC(O)CO)c2c(=O)[nH]1 ; Nc1nc2[nH]cc(CNC[C@H](O)CO)c2c(=O)[nH]1 ; Nc1nc2[nH]cc(CNC[C@@H](O)CO)c2c(=O)[nH]1 ; Nc1nc2[nH]cc(CNCCCc3ccccc3)c2c(=O)[nH]1]

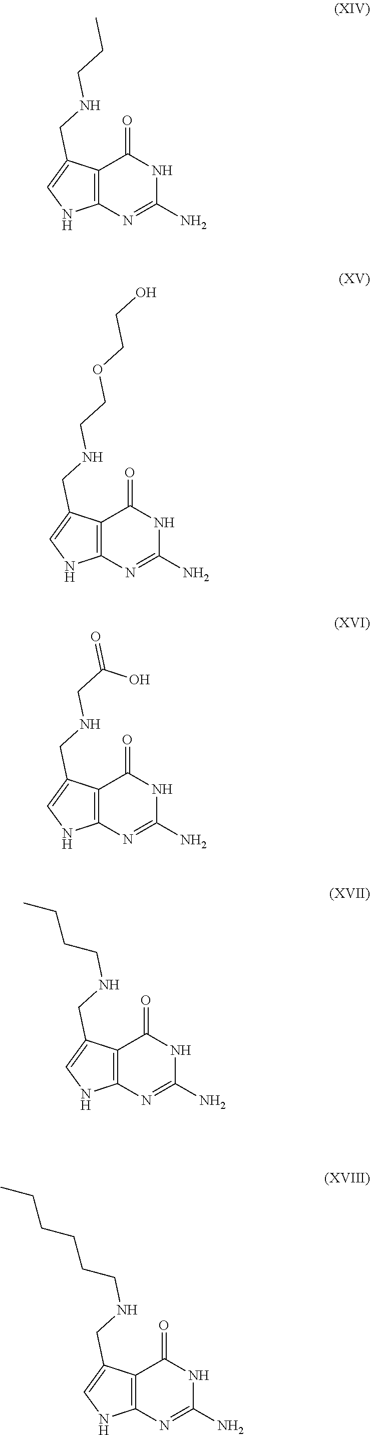 [SMILES: CCCNCc1c[nH]c2nc(N)[nH]c(=O)c12 ; Nc1nc2[nH]cc(CNCCOCCO)c2c(=O)[nH]1 ; Nc1nc2[nH]cc(CNCC(=O)O)c2c(=O)[nH]1 ; CCCCNCc1c[nH]c2nc(N)[nH]c(=O)c12 ; CCCCCCNCc1c[nH]c2nc(N)[nH]c(=O)c12]

The compound of formula (VIIa) is epoxyqueuine, also known as 7-(5-[3,4-epoxy-2,5-dihydroxycyclopent-1-yl)amino]methyl)-7-deazaguanine.

The compound of formula (VIIIa) is queuosine also known as 2-amino-5-({[(1S,4S,5R)-4,5-dihydroxycyclopent-2-en-1-yl]amino}methyl)-7-(β-D-ribofuranosyl)-1,7-dihydro-4H-pyrrolo[2,3-d]pyrimidin-4-one.

The compound of formula (IXa) is epoxyqueuosine also known as 5 7-(5-[(3,4-epoxy-2,5-dihydroxycyclopent-1-yl)amino]methyl)-7-deazaguanosine.

Preferably, the compound of formula (II) according to the invention is selected from the group consisting of mannosyl-queuine, galactosyl-queuine, glutamyl-queuine, galactosyl-queuosine, mannosyl-queuosine, glutamyl-queuosine, queuine-tRNA, and epoxyqueuine-tRNA.

The compound of formula (XIII) is N-((2-amino-4-oxo-4,7-dihydro-3Hpyrrolo[2,3-d]pyrimidin-5-yl)methyl)-3-phenylpropan-1-amine.

The compound (XIV) is N-((2-amino-4-oxo-4,7-dihydro-3Hpyrrolo[2,3-d]pyrimidin-5-yl)methyl)-propan-1-amine.

The compound (XVII) is N-((2-amino-4-oxo-4,7-dihydro-3Hpyrrolo[2,3-d]pyrimidin-5-yl)methyl)-butan-1-amine.

The compound (XVIII) is N-((2-amino-4-oxo-4,7-dihydro-3Hpyrrolo[2,3-d]pyrimidin-5-yl)methyl)-hexan-1-amine.

Preferably also the compound of formula (I), in particular the compound of formula (II), according to the invention is selected from the group consisting of queuine-tRNA^{Asp}, queuine-tRNA^{Tyr}, epoxyqueuine-tRNA^{Asp}, epoxyqueuine-tRNA^{Tyr}, queuine-tRNA^{Asn}, queuine-tRNA^{His}, epoxyqueuine-tRNA^{Asn}, epoxyqueuine-tRNA^{His}, mannosyl-queuine-tRNA^{Asp}, galacotsyl-queuine-tRNA^{Tyr}, mannosyl-epoxyqueuine-tRNA^{Asp}, and galacotsyl-epoxyqueuine-tRNA^{Tyr}.

Most preferably also the compound of formula (I), in particular the compound of formula (II), according to the invention is selected form the group consisting of queuine, ent-queuine, queuosine, epoxyqueuine, epoxyqueuosine, mannosyl-queuine, galactosyl-queuine, glutamyl-queuine, galactosyl-queuosine, mannosyl-queuosine, glutamyl-queuosine, queuine-tRNA and epoxyqueuine-Trna, a compound of formula (XII), (XIIa) and (XIIb).

### Alzheimer's Disease

Alzheimer's disease is well known of one of skilled in the art. It is notably defined by class G30 of the 10th revision of the International Classification of Diseases (ICD-10) 2016 version set by the World Health Organization. In addition, Alzheimer's disease is defined by the following diagnostic criteria in the fifth edition of the Diagnostic and Statistical Manual of Mental Disorder (DSM-5) (2013) American Psychiatric Association, pages 611-614:

A. The criteria are met for major or mild neurocognitive disorder.
B. There is insidious onset and gradual progression of impairment in one or more cognitive domains (for major neurocognitive disorder, at least two domains must be impaired).
C. Criteria are met for either probable or possible Alzheimer's disease as follows:

For Major Neurocognitive Disorder:
Probable Alzheimer's disease is diagnosed if either of the following is present; otherwise, possible Alzheimer's disease should be diagnosed.
1. Evidence of a causative Alzheimer's disease genetic mutation from family history or genetic testing.
2. All three of the following are present:
   a. Clear evidence of decline in memory and learning and at least one other cognitive (based on detailed history or serial neuropsychologicaltesting).
   b. Steadily progressive, gradual decline in cognition, without extended plateaus.
   c. No evidence of mixed etiology (i.e. absence of other neurodegenerative or cerebrovascular disease, or another neurological, mental, or systemic disease or condition likely contributing to cognitive decline).

For Mild Neurocognitive Disorder:
Probable Alzheimer's disease is diagnosed if there is evidence of a causative Alzheimer's disease genetic mutation from either genetic testing or family history.
Possible Alzheimer's disease is diagnosed if there is no evidence of a causative Alzheimer's disease genetic mutation from either genetic testing or family history, and all three of the following are present:
1. Clear evidence of decline in memory and learning.
2. Steadily progressive, gradual decline in cognition, without extended plateaus.
3. No evidence of mixed etiology (i.e. absence of other neurodegenerative or cerebrovascular disease, or another neurological or systemic disease or condition likely contributing to cognitive decline).
   D. The disturbance is not better explained by cerebrovascular disease, another neurodegenerative disease, the effects of a substance, or another mental, neurological, or systemic disorder.

Alzheimer's disease prevented or treated according to the invention may in particular be Alzheimer's disease at an early stage, Alzheimer's disease at an intermediate stage, Alzheimer's disease at an advanced stage, pre-clinical Alzheimer's disease, a dementia due to Alzheimer's disease, a mild cognitive disorder due to Alzheimer's disease, a mild neurocognitive disorder due to Alzheimer's disease, a major neurocognitive disorder due to Alzheimer's disease, the probable Alzheimer's disease, or the possible Alzheimer's disease.

Preferably, the prevention or treatment of Alzheimer's disease according to the invention relates to the prevention or treatment of at least one cognitive or neurocognitive disorder due to Alzheimer's disease, in particular selected from the group consisting of memory disorder or a learning disability.

The invention also relates to the prevention or treatment of symptoms of Alzheimer's disease.

### Individual

The individual according to the invention is preferably a human.

In a preferred embodiment of the invention, the individual according to the invention is 65 years old or more. In another preferred embodiment of the invention, the individual according to the invention is less than 65 years old.

The individual according to the invention may present one or more symptoms of dementia or may have dementia.

The individual according to the invention may not have dementia. In particular, the individual according to the invention may have cognitive disorders, in particular mild cognitive disorders corresponding to the Anglo-Saxon denomination Mild Cognitive Impairment (MCI), which is well known of one of skilled in the art, and notably defined by Petersen et al. (1999) Arch Neurol 56:303-308. An individual is generally defined as having a MCI in the event of a subjective complaint associated with objective evidence of a deficit in memory performance with a saving of the cognitive and overall intellectual functioning and integrity of activities of everyday life. Preferably, an individual with a MCI according to the invention has a Mini Mental State Examination (MMSE) score, in particular in the consensus version of the Groupe de Réflexion sur les Evaluation COgnitives (GRECO) (Study on Group of cognitive evaluation), higher than the score corresponding to the 5th percentile, according to its age and its socio-cultural level.

Besides, the individual according to the invention may also not have cognitive disorder.

### Additional Compound

The additional compound useful for the prevention or treatment of Alzheimer's disease according to the invention can be of any type known of one of skilled in the art. Preferably, the additional compound according to the invention is selected from the group consisting of donepezil, galantamine, memantine, and rivastigmine.

Preferably, the additional compound in the dietary supplement is selected from the group consisting of vitamins, minerals, fatty acids, amino acids, antioxidants and derivatives or precursors thereof.

Preferably vitamins are selected from the group consisting of pyridoxine, pyridoxal phosphate (Vitamin B₆), riboflavin, thiamine, vitamin E, vitamin K3, vitamin C, niacin, CoQ10 and β-carotene.

Preferably, minerals are selected from the group consisting of calcium, magnesium, selenium and phosphorus.

Preferably, the amino-acid is L-DOPA (levodopa).

Preferably, the fatty acids are selected from the group consisting of Levo-carnitine and acetyl-L-carnitine.

### Administration

As intended herein, “combined” or “in combination” means that the compound of formula (I), in particular the compound of formula (II) as defined above, is administered at the same time than another compound or product, either together, i.e. at the same administration site, or separately, or at different times, provided that the time period during which the compound of formula (I) as defined above exerts its effects on the individual and the time period during which the additional agent or product exerts its pharmacological effects on the individual, at least partially intersect.

Preferably also, the compound of formula (I), in particular the compound of formula (II), according to the invention or the pharmaceutically acceptable salt or hydrate thereof is for an administration or is administered at a dosage regimen of from 0.01 to 40 mg/kg/d, more preferably of from 0.01 to 10 mg/kg, even more preferably of from 0.01 to 1 mg/kg/d, and most preferably of from 0.01 to 0.1 mg/kg/d.

Preferably, the compound of formula (I), in particular the compound of formula (II) according to the invention or the pharmaceutically acceptable salt or hydrate thereof is in a form suitable for an administration or is administered by the oral route, the intradermal route, the intravenous route, the intramuscular route or the subcutaneous route. Preferably, the compound of formula (I), in particular the compound of formula (II), according to the invention, or the pharmaceutical composition, medicament, products or dietary supplement comprising it is in a form suitable for an administration or is administered by a hypodermic implant.

Preferably, the compound of formula (I) according to the invention, or the pharmaceutical composition, medicament, products or dietary supplement comprising it is in the form of a powder, sachets, tablets, gelatine, capsules, or a liquid or gel solution.

Preferably also, the pharmaceutical composition, medicament, products or dietary supplement according to the invention, comprises the compound of formula (I) according to the invention, in particular queuine, ent-queuine, queuosine, the compound of formula (XII), (XIIa), or (XIIb) at a unit dose of at least 0.15 mg, 1 mg, 10 mg, 50 mg, 100 mg, 500 mg or 1000 mg.

Preferably also, the pharmaceutical composition, medicament, products or dietary supplement according to the invention, comprises an extract, in particular a purified extract, from microorganism and/or plant, which comprises the compound of formula (I) according to the invention, in particular queuine, ent-queuine, queuosine, the compound of formula (XII), (XIIa), or (XIIb) in particular at a unit dose of at least 0.15 mg, 1 mg, 10 mg, 50 mg, 100 mg, 500 mg or 1000 mg.

The invention will be explained in more detailed in the following non-limitative Example.

### DESCRIPTION OF THE FIGURES

FIG. 1

FIG. 1 shows the effect of queuine at 5 different concentrations (30 nM, 100 nM, 300 nM, 1 μM, and 3 μM) on cortical survival, expressed in percentage of vehicle group, after intoxication with Aβ₁₋₄₂. Each value represents the mean±sem (100%=no ABI-42). (*) represents p<0.05. BDNF (50 ng/ml) is the referenced compound.

FIG. 2

FIG. 2 shows the effect of queuine at 5 different concentrations (30 nM, 100 nM, 300 nM, 1 μM, and 3 μM) on the neurite network, expressed in percentage of vehicle group, after intoxication with Aβ₁₋₄₂. Each value represents the mean±sem (100%=no A$1-42). (*) represents p<0.05. BDNF (50 ng/ml) is the referenced compound.

FIG. 3

FIG. 3 show the effect of queuine at 5 different concentrations (30 nM, 100 nM, 300 nM, 1 μM, and 3 μM) on the ratio Tau/neurite, expressed in percentage of vehicle group, after intoxication with Aβ₁₋₄₂. Each value represents the mean±sem (100%=no A$1-42). (*) represents p<0.05. BDNF (50 ng/ml) is the referenced compound.

### EXAMPLE 1: EVALUATION OF THE EFFECT OF COMPOUNDS ACCORDING TO THE INVENTION IN AN IN VITRO MODEL OF ALZHEIMER'S DISEASE

The purpose of this example is to evaluate the effects of compounds according to the invention in the in vitro model of Alzheimer's disease deriving from intoxication of cortical neurons by the amyloid β₁₋₄₂ peptide, in accordance with Callizot et al. (2013) J. Neurosci. Res. 91: 706-16; Chumakov et al. (2015) Nature Scientific Reports 5: 7608; Combs et al. (2000) J. Neurosci. 20:558-67; Cummings et al. (1998) Neurology. 51(Suppl 1): S2-17, discussion S65-7; Harrison (1990) J. Physiol. 422: 433-446; Kawahara et al. (2000) Brain Res. Bull. 53: 389-97; Pike et al. (1991) Brain Res. 563: 311-4; Sakono et al. (2010) FEBS J. 277: 1348-58; Singer et al. (1999) J. Neurosci. 19: 2455-2463; Sisodia et al. (1990) Science. 248: 492-5.

A. Materials and Methods

1. Cortical Neuron Culture Rat cortical neurons are cultured as described by Singer et al. (1999) J. Neurosci. 19: 2455-2463. Briefly pregnant female rats of 15 days gestation are killed by cervical dislocation and the foetuses are removed from the uterus. The cortex is removed and placed in ice-cold medium of Leibovitz containing 2% of Penicillin and Streptomycin (PS) and 1% of bovine serum albumin. The cortex is dissociated by trypsinisation (0.05%) for 20 min at 37° C. The reaction is stopped by the addition of Dulbecco's modified Eagle's medium (DMEM) containing DNAase I grade II (0.1 mg/ml) and 10% of foetal calf serum (FCS). Cells are then mechanically dissociated by 3 passages through a 10 ml pipette. Cells are then centrifuged at 515×g for 10 min at 4° C. The supernatant is discarded and the cells of pellet are re-suspended in a defined culture medium consisting of Neurobasal supplemented with 2% of B27 supplement, 2 mM of L-glutamine, 2% of PS solution and 10 ng/ml of BDNF. Viable cells are counted in a Neubauer cytometer using the trypan blue exclusion test. The cells are seeded at a density of 30 000 cells/well in 96 well-plates pre-coated with poly-D-lysine and are cultured at 37° C. in a humidified air (95%)/CO2 (5%) atmosphere.

2. Effect of Queuine on Neuronal Cell Death after β-Amyloid Injury in Cortical Neurons

2.1. Incubation of Queuine 6 Days Before Intoxication

After 5 days of culture, the cells are incubated with queuine (7 concentrations). After 6 days of incubation with queuine, cells are intoxicated with 10 μM of amyloid 1-42 oligomers in a medium as defined by Callizot et al. (2013) J. Neurosci. Res. 91: 706-16 in the presence of queuine. BDNF (50 ng/ml) is used as positive control and reference compound.

The following conditions are tested:
Vehicle solution
Amyloid β₁₋₄₂ at 10 μM, 24h
Amyloid β₁₋₄₂ at 10 μM, 24h+BDNF (50 ng/mL)
Amyloid-β₁₋₄₂ at 10 μM, 24h+queuine (0.0001 μM; 0.001 μM; 0.01 μM; 0.03 μM; 0.100 μM; 0.300 μM; 1 μM) added 6 days before intoxication.

Six wells per condition and 3 cultures are performed.

2.2. Incubation of Queuine 24H Before the Intoxication

Briefly, on day 10, queuine (7 concentrations) is added. 24H after, cells are intoxicated with Amyloid β₁₋₄₂ at 10 μM for 24h.

The following conditions are tested:
Control medium
Amyloid β₁₋₄₂ at 10 μM, 24h
Amyloid β₁₋₄₂ at 10 μM, 24h+BDNF (50 ng/mL)
Amyloid β₁₋₄₂ at 10 μM, 24h+queuine (0.001 μM; 0.01 μM; 0.03 μM; 0.100 μM; 0.300 μM; 1 μM; 3 μM) added 24H before intoxication.

Six wells per condition and 3 cultures are performed.

3. End Point Evaluation: Measure of Total Number of Rat Cortical Neurons

24 hours after intoxication, cells are fixed by a cold solution of ethanol (95%) and acetic acid (5%) for 5 min. The cells are then permeabilized and non-specific sites are blocked with a solution of phosphate buffered saline (PBS) containing 0.1% of saponin and 1% fetal calf serum (FCS) for 15 min at room temperature. Then, cells are incubated with monoclonal antibody anti microtubule-associated-protein 2 (MAP-2).

This antibody stains specifically cell bodies and neurites of neurons. This antibody is revealed with Alexa Fluor 488 goat anti-mouse IgG. Nuclei of neurons are labeled by a fluorescent marker (Hoechst solution).

The neuron survival is evaluated (number of MAP-2 positive neuronal cell bodies are counted).

For each well of culture, 20 pictures per well are taken using InCell Analyzer™ 2000 (GE Healthcare) with 20× magnification. All the images are taken in the same conditions. All values are expressed as mean±s.e. mean. Statistical analyses are done on different conditions (ANOVA followed by Dunnett's test).

An increased survival of neurons incubated with queuine as compared to conditions without queuine indicates that queuine has anti-Alzheimer's disease properties.

### EXAMPLE 2: IN VIVO EVALUATION OF THE EFFECT OF COMPOUNDS ACCORDING TO THE INVENTION IN A MURINE MODEL OF ALZHEIMER'S DISEASE BY ADMINISTRATION OF THE AMYLOID B25-35 PEPTIDE

The purpose of this example is the in vivo evaluation of compounds according to the invention in a murine model of Alzheimer's disease by administration of the amyloid $25-35 peptide in accordance with Maurice et al. (1996) Brain Res. 706:181-193; Maurice et al. (1998) Neuroscience 83:413-428; Meunier et al. (2006) J. Pharmacol. Exp. Ther. 317:1307-1319; Meunier et al. (2013) Genome Research 23:34-45; Villard et al. (2009) Neurophsychopharmacologie 34:1552-1566; Villard et al. (2011) J. Psychopharmacol. 25:1101-1117.

A. Materials and Methods

1. Animals

Male Swiss mice, 5 weeks old and weighing 30-35 g (JANVIER, Saint Berthevin, France), are kept for housing. Animals are housed in groups with access to food and water ad libitum, except during behavioural experiments. They are kept in a temperature and humidity-controlled animal facility on a 12 h/12 h light/dark cycle (lights off at 07:00 pm). Mice are numbered by marking their tail using permanent markers.

2. Protocol

Seventy-two male Swiss mice (30-35 g) are used. Six animal groups are constituted and submitted to different treatments:

TABLE 1
treatment groups

| | Number of mice |
|---|---|
| 1. Sc.Aβ + vehicle solution, IP | 12 |
| 2. Aβ₂₅₋₃₅ + vehicle, IP | 12 |
| 3. Aβ₂₅₋₃₅ + reference compound | 12 |
| (Donepezil, 1 mg/kg), IP | |
| 4. Aβ₂₅₋₃₅ + queuine, IP (1 mg/Kg) | 12 |
| 5. Aβ₂₅₋₃₅ + queuine, IP (5 mg/Kg) | 12 |
| 6. Aβ₂₅₋₃₅ + queuine, IP (30 mg/Kg) | 12 |
| Total mice number | 72 |

Day 1: a scrambled version of the amyloid $25-35 peptide (Sc.Aβ, negative control) or the amyloid β₂₅₋₃₅ peptide (Aβ25-35) is injected intracerebroventricularly (ICV) (the Aβ₂₅₋₃₅ peptide causes cellular intoxication). Between the 1st and 10th day, treatments are administered once a day intraperitoneally (IP).

At days 8 to 10, two different behavioral tests are used:
Spontaneous alternation procedure in the Y-maze (assessing spatial working memory) at day 8 (7 days after the peptide injection);
Passive avoidance response (assessing contextual long-term memory) with training at day 9 and retention session at day 10.

On day 10, after the behavioral test, animals are sacrificed by decapitation. Blood plasma samples are collected on each animal. The hippocampus and frontal cortex are dissected out and frozen in liquid nitrogen.

On n=6 animals per group, one hemi-hippocampus is used to determine the lipid peroxidation levels using a colorimetric method.

On n=6 animals per group, one hemi-hippocampus is used to determine the level of four biochemical markers using ELISA assays.

The other brain structures are stored at −80° C. and are available for supplementary biochemical assays.

3. Products

3.1. Compounds

Donepezil is solubilized in NaCl 0.9%
Denomination: donepezil
IUPHAR name: 2-[(1-benzyl-4-piperidyl)methyl]-5,6-dimethoxy-2,3-dihydroinden-1-one, hydrochloride, hydrate (1:1:1)
CAS: 120014-06-4
Supplier: Sigma-Aldrich (France)
Reference: D6821

Aβ₂₅₋₃₅:
Denomination: amyloid-B protein (25-35), human, mouse, rat
CAS: 131602-53-4
Supplier: Polypeptides (France)
Reference: SC489

Sc.Aβ:
Denomination: scrambled amyloid-B protein (25-35), human, mouse, rat
CAS: NA
Supplier: Polypeptides (France)
Reference: SC942

Queuine is obtained from Synthenova SAS, 15 rue J. Baptiste Lamarck, 14 200 Herouville Saint Clair.

3.2. Amyloid Peptides Administration

Each mouse is anesthetized with isoflurane 2.5% and injected ICV with Aβ₂₅-3₅ peptide (9 nmol/mouse) or Sc.Aβ peptide (9 nmol/mouse), in a final volume of 3 L/mouse. These injections help to establish a mouse model of Alzheimer's disease according to Maurice et al. (1996) Brain Res. 706:181-193; Maurice et al. (1998) Neuroscience 83:413-428; Meunier et al. (2006) J. Pharmacol. Exp. Ther. 317:1307-1319; Meunier et al. (2013) Genome Research 23:34-45; Villard et al. (2009) Neurophsychopharmacologie 34:1552-1566; Villard et al. (2011) J. Psychopharmacol. 25:1101-1117.

4. Conduct of the Tests

4.1. Behavioral and Biochemical Analysis

4.1.1. Spontaneous Alternation Performance

On day 8, all animals are tested for spontaneous alternation performance in the Y-maze, an index of spatial working memory. The Y-maze is made of grey polyvinylchloride. Each arm is 40 cm long, 13 cm high, 3 cm wide at the bottom, 10 cm wide at the top, and converging at an equal angle. Each mouse is placed at the end of one arm and allowed to move freely through the maze during an 8 min session. The series of arm entries, including possible returns into the same arm, is checked visually. An alternation is defined as entries into all three arms on consecutive occasions. The number of maximum alternations is therefore the total number of arm entries minus two and the percentage of alternation is calculated as (actual alternations/maximum alternations)×100. Parameters included the percentage of alternation (memory index) and total number of arm entries (exploration index) (Maurice et al. (1996) Brain Res. 706:181-193; Maurice et al. (1998) Neuroscience 83:413-428; Meunier et al. (2006) J. Pharmacol. Exp. Ther. 317:1307-1319; Meunier et al. (2013) Genome Research 23:34-45; Villard et al. (2009) Neurophsychopharmacologie 34:1552-1566; Villard et al. (2011) J. Psychopharmacol. 25:1101-1117). Animals showing an extreme behavior (Alternation percentage <20% or >90% or number of arm entries <10) are discarded from the calculation.

4.1.2. Passive Avoidance Test

The apparatus is a two-compartments (15×20×15 cm high) box with one illuminated with white polyvinylchloride walls and the other darkened with black polyvinylchloride walls and a grid floor. A guillotine door separates each compartment. A 60 W lamp positioned 40 cm above the apparatus lights up the white compartment during the experiment. Scrambled footshocks (0.3 mA for 3 s) are delivered to the grid floor using a shock generator scrambler (Lafayette Instruments, Lafayette, USA). The guillotine door is initially closed during the training session. During training session, each mouse is placed into the white compartment. After 5 s, the door is raised. When the mouse entered the dark compartment and placed all its paws on the grid floor, the door is closed and the footshock delivered for 3 s. The step-through latency, that is, the latency spent to enter the dark compartment, and the number of vocalizations are recorded. The retention test is carried out 24 h after training. Each mouse is placed again into the white compartment. After 5 s, the door is raised. The step-through and escape latencies (corresponding to the re-exit from the dark compartment) are recorded up to 300 s (Meunier et al. (2006) J. Pharmacol. Exp. Ther. 317:1307-1319; Villard et al. (2009) Neurophsychopharmacologie 34:1552-1566; Villard et al. (2011) J. Psychopharmacol. 25:1101-1117).

Animals that showed latencies during the training and retention session both lower than 10 s are considered as failing to respond to the procedure and are discarded from the calculations.

4.2. Lipid Peroxidation Measurement

All mice from each group are sacrificed by decapitation and both hippocampi are rapidly removed, weighed and kept in liquid nitrogen until assayed. After thawing, one hippocampus per mice is homogenized in cold methanol (1/10 w/v), centrifuged at 1,000 g during 5 min and the supernatant placed in Eppendorf tube. The reaction volume of each homogenate is added to FeSO4 1 mM, H2SO4 0.25 M, xylenol orange 1 mM and incubated for 30 min at room temperature. After reading the absorbance at 580 nm (A5801), 10 μl of cumene hydroperoxide (CHP) 1 mM are added to the sample and incubated for 30 min at room temperature, to determine the maximal oxidation level. The absorbance is measured at 580 nm (A5802). The level of lipid peroxidation is determined as CHP equivalents according to: CHPE=A₅₈₀1/A₅₈₀2×[CHP (nmol)] and expressed as CHP equivalents per mg of tissue and as percentage of control group data (V-treated Sc.Aβ-administered mice).

4.3. ELISA Assays

Contents in Bax, Bcl2, GFAP and Caspase-3 are analyzed by ELISA assays.

The kits are:

Mitochondrial Dysfunction/Apoptosis

Caspase-3: Supplier: USCNK Reference: SEA626Mu

Bax: Supplier: USCNK Reference: SEB343Mu

Bcl2: Supplier: USCNK Reference: SEA778Mu

Inflammatory Processes

GFAP: Supplier: USCNK Reference: SEA425Mu

For all assays, the cortices are homogenized after thawing in a Tris buffer (50 mM Tris, 150 mM NaCl, pH 7.5) and sonicated for 20 s. After centrifugation (16,100 g for 15 min, 4° C.), supernatants are used for ELISA assays according to the manufacturer instructions. For each assay, absorbance is read at 450 nm and sample concentration is calculated using the standard curve. Results are expressed in pg or ng of marker per mg of tissue and in percent of Sc.Aβ+Vehicle solution. 6 samples per group (n=36/ELISA kit) are assayed in duplicates.

An improvement of the tested behavioral and biochemical characteristics of mice administered with queuine as compared to mice which did not receive queuine indicates that queuine has anti-Alzheimer's disease properties.

### EXAMPLE 3: EVALUATION OF THE EFFECT OF COMPOUNDS ACCORDING TO THE INVENTION IN AN IN VITRO MODEL OF ALZHEIMER'S DISEASE

The purpose of this example is to evaluate the effects of compounds according to the invention in the in vitro model of Alzheimer's disease deriving from intoxication of cortical neurons by the amyloid β₁₋₄₂ peptide, in accordance with Callizot et al. (2013) J. Neurosci. Res. 91: 706-16; Chumakov et al. (2015) Nature Scientific Reports 5: 7608; Combs et al. (2000) J. Neurosci. 20:558-67; Cummings et al. (1998) Neurology. 51(Suppl 1): S2-17, discussion S65-7; Harrison (1990) J. Physiol. 422: 433-446; Kawahara et al. (2000) Brain Res. Bull. 53: 389-97; Pike et al. (1991) Brain Res. 563: 311-4; Sakono et al. (2010) FEBS J. 277: 1348-58; Singer et al. (1999) J. Neurosci. 19: 2455-2463; Sisodia et al. (1990) Science. 248: 492-5, Jan et al., (2010) Nat. Protoc., 5: 1186-1209.

A. Material and Method

1. Cortical Neurons

Rat cortical neurons are cultured as described by Callizot et al., (2013) with modifications. Briefly pregnant female rat (Wistar) of 15 days of gestation are killed. Foetuses are collected and immediately placed in ice-cold L15 Leibovitz medium with a 2% penicillin (10,000 U/mL) and streptomycin (10 mg/ml) solution (PS) and 1% bovine serum albumin (BSA). Cortex are treated for 20 min at 37° C. with a trypsin- EDTA solution at a final concentration of 0.05% trypsin and 0.02% EDTA. The dissociation is stopped by addition of Dulbecco's modified Eagle's medium (DMEM) with 4.5 g/L of glucose, containing DNAse I grade II (final concentration 0.5 mg/ml) and 10% fetal calf serum (FCS). Cells are mechanically dissociated by three forced passages through the tip of a 10-ml pipette. Cells are then centrifuged at 515×g for 10 min at 4° C. The supernatant is discarded, and the pellet is resuspended in a defined culture medium consisting of Neurobasal medium with a 2% solution of B27 supplement, 2 mmol/litter of L-glutamine, 2% of PS solution, 10 ng/mL of brain-derived neurotrophic factor (BDNF), 2% of heat-inactivated horse serum, 2% of heat-inactivated FCS, 1 g/L of glucose, 1 mM of sodium pyruvate, and 100 μM of non-essential amino acids. Viable cells are counted in a Neubauer cytometer, using the trypan blue exclusion test. The cells are seeded at a density of 45,000 per well in 96-well plates (for immunostaining) precoated with poly-L-lysine and are cultured at 37° C. in an air (95%)-CO2 (5%) incubator. For 96 wells plates, only 60 wells are used. The wells of first and last lines and columns are not used (to avoid the edge effect) and are filled with sterile water. The medium is changed every 2 days.

2. Test Compound and Human Aβ₁₋₄₂Exposure

On day 10 of culture, queuine is dissolved in the culture medium and pre-incubated with primary cortical neurons for 24H, before Aβ₁₋₄₂ exposure. Then on day 11 of culture, the cortical neurons are intoxicated with AB solutions. The Aβ₁₋₄₂ preparation is done following the procedure described by Callizot et al., (2013). Briefly, Aβ₁₋₄₂ peptide are dissolved in the defined culture medium mentioned above, at an initial concentration of 40 μmol/L. This solution is gently agitated for 3 days at 37° C. in the dark and immediately used after being properly diluted in culture medium to the concentration used (5 μM, 0.5μM oligomers).

Aβ₁₋₄₂ preparation is added to a final concentration of 5μM (0.5 μM oligomers, ABO) diluted in control medium in presence of queuine, for 72 hours.

3. Organization of Culture Plates

Each compound is tested on 1 culture in a 96 well plate (6 wells per conditions). For 96 wells plates, only 60 wells are used. The wells of first and last lines and columns are not used (to avoid the edge effect) and are filled with sterile water. Queuine is added 24h before Aβ₁₋₄₂ application. The following conditions are assessed:

Plate 1 (MAP-2/Tau phospho/OX-41)
Control (vehicle)
+ Aβ₁₋₄₂ (5 μM 72 H)/vehicle
+ Aβ₁₋₄₂ (5 μM 72 H)/queuine (30 nM)
+ Aβ₁₋₄₂ (5 μM 72 H)/queuine (100 nM)
+ Aβ₁₋₄₂ (5 μM 72 H)/queuine (300 nM)
+ Aβ₁₋₄₂ (5 μM 72 H)/queuine (1 μM)
+ Aβ₁₋₄₂ (5 μM 72 H)/queuine (3 μM)
+ Aβ₁₋₄₂ (5 μM 72 H)/BDNF (50 ng/mL)

4. Evaluation

4.1. Immunostaining

72 hours after Aβ₁₋₄₂ application, the cell culture supernatant are collected cytokine quantification (e.g. TNFα) and the cortical neurons are fixed by a cold solution of ethanol (95%) and acetic acid (5%) for 5 min at −20° C. After permeabilization with 0.1% of saponin, cells are incubated for 2 hours with:
a) chicken polyclonal antibody antimicrotubule-associated-protein 2 (MAP-2) at dilution of 1/1000 in PBS containing 1% fetal calf serum and 0.1% of saponin (this antibody stains specifically cell bodies and neurites, allowing study of neuronal cell death and neurite network).
b) mouse monoclonal anti-tau phosphor (Thr212, Ser214) at the dilution of 1/100 in PBS containing 1% foetal calf serum and 0.1% of saponin.
c) rabbit polyclonal antibody anti-SIRP alpha/CD172a at dilution of 1/400 in PBS containing 1% fetal calf serum and 0.1% of saponin (this antibody stains specifically the microglia and is allow to evaluate the activation of the microglial cells).

These antibodies are revealed with secondary antibodies goat anti-rabbit IgG, goat anti-mouse IgG and goat anti-chicken IgG coupled with an Alexa Fluor at the dilution 1/400 in PBS containing 1% FCS, 0.1% saponin, for 1 hour at room temperature.

For each condition, 30 pictures/well (representative of the whole well area) are automatically taken using ImageXpress (Molecular Devices) at 20× magnification. All images are automatically acquired under the same conditions. The following analysis is performed with Custom Module Editor (Molecular Devices):
total neuron survival (number of MAP-2 positive neuron)
total neurite network (length of MAP-2 positive neurite)
hyperphosphorylation of Tau in neuron cytoplasm (AT100, overlapping Tau/MAP-2, μm² of overlapping)
total microglia activation (area of microglial cells, μm² of OX-41 staining).

4.2 TNF-α Evaluation

In order to assess the activation of microglia in the cell culture, the levels of TNF-α are quantified in cell culture supernatant after 72h (end of the culture) by ELISA.

5. Statistical Analysis

All values are expressed as mean+/−SEM. Statistical analysis are performed by one-way ANOVA, followed by Dunnett's or a PLSD Fisher test, p<0.05 is considered significant.

6. Results

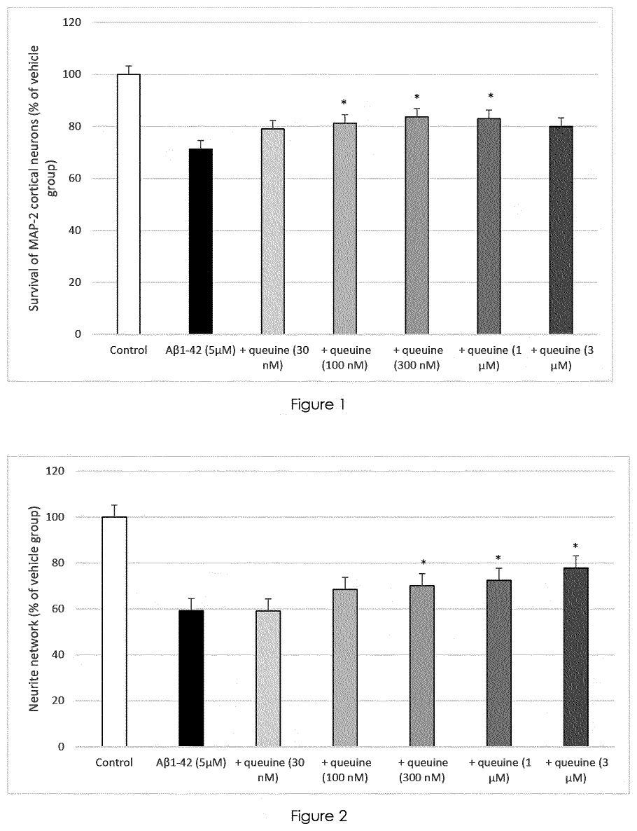

FIG. 1 shows that the number of cortical neurons decreases significantly in the presence of the peptide A$1-42 (5 μM), compared to the control conditions. BDNF (50 ng/ml) restores the level neuronal staining for MAP-2.

Queuine has a neuroprotective effect on cortical neurons intoxicated with Aβ₁₋₄₂ peptide. Indeed, queuine significantly restores the survival of neurons at 100 nM (*, p <0.05), 300 nM (*, p<0.05) and 1 μM (*, p<0.05).

FIG. 2 shows that the neurite network decreases in the presence of the peptide Aβ₁₋₄₂ (5 μM) compared to the control conditions. BDNF (50 ng/ml) restores the neurite network. Queuine has a neuroprotective effect on neurons intoxicated with Aβ₁₋₄₂ peptide. Indeed, queuine significantly restores the neurite network at 300 nM (*, p <0.05), 1 μM (*, p<0.05) and 3 μM (*, p<0.05).

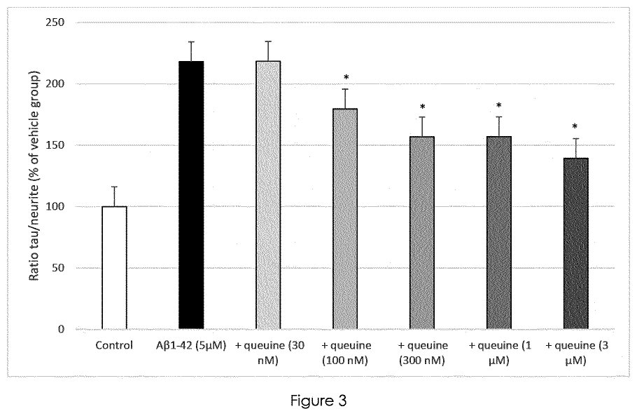

FIG. 3 shows that the peptide Aβ₁₋₄₂ (5 μM) induces a significant increase in Tau hyperphosphorylation. BDNF (50 ng/ml) decreases Tau hyperphosphorylation. Queuine at 100 nM (*, p<0.05), 300 nM (*, p<0.05), and 1 μM (*, p<0.05) and 3 μM (*, p<0.05) restores the level of hyperphosphorylation of Tau.

The results of the TNF-α evaluation are shown in the in following Table 1:

TABLE 1
TNFa release in a culture of cortical neurons and microglia after Aβ₁₋₄₂ injury (5 μM, 72 H)

| | | Aβ₁₋₄₂ | Aβ₁₋₄₂ (5 μM) + | Aβ₁₋₄₂ (5 μM) + | Aβ₁₋₄₂ (5 μM) + | Aβ₁₋₄₂ (5 μM) + |
|---|---|---|---|---|---|---|
| | Control | (5 μM) | queuine (300 nM) | queuine (1 μM) | queuine (3 μM) | BDNF (50 ng/ml) |
| TNFa (% of | 100.00 | 126.75 | 105.54 | 87.33 | 93.32 | 102.14 |
| vehicle group) | | | | | | |

Table 1 shows that the peptide Aβ₁₋₄₂ (5 μM) induces a significant increase in TNFα. BDNF (50 ng/ml) decreases the level of TNFα. Queuine at 300 nM (*, p<0.05), 1 μM (*, p<0.05), and 3 μM (*, p<0.05) restores the level of TNFα.

1-16. (canceled)

17. A method for treating or preventing Alzheimer's disease in an individual comprising administering to the individual queuine or a pharmaceutically acceptable salt or hydrate thereof.

18. The method of claim 17, wherein the queuine or pharmaceutically acceptable salt or hydrate thereof is administered at a unit dose of from 5 mg/kg to 1,500 mg/kg.

19. The method of claim 17, wherein the queuine or pharmaceutically acceptable salt or hydrate thereof is administered in a dosage regimen of from 0.01 mg/kg/d to 40 mg/kg/d.

20. The method of claim 17, wherein the queuine or pharmaceutically acceptable salt or hydrate thereof is administered orally, intradermally, intravenously, intramuscularly, or subcutaneously.

21. The method of claim 17, wherein the queuine or pharmaceutically acceptable salt or hydrate thereof is administered in combination with at least one additional compound useful for treating or preventing Alzheimer's disease.

22. The method of claim 21, wherein the additional compound is selected from the group consisting of donepezil, galantamine, memantine, and rivastigmine.

23. A composition comprising (i) queuine or a pharmaceutically acceptable salt or hydrate thereof and (ii) an additional compound useful for treating or preventing Alzheimer's disease.

24. The composition of claim 23, further comprising a pharmaceutically acceptable excipient or vehicle.

25. The composition of claim 23, wherein the additional compound is selected from the group consisting of donepezil, galantamine, memantine, and rivastigmine.

26. The composition of claim 25, further comprising a pharmaceutically acceptable excipient or vehicle.

27. A method for causing a neuroprotective effect in an individual having Alzheimer's disease comprising administering to the individual queuine or a pharmaceutically acceptable salt or hydrate thereof.

28. The method of claim 27, wherein the queuine or pharmaceutically acceptable salt or hydrate thereof is administered in a dosage regimen of from 0.01 mg/kg/d to 40 mg/kg/d.

29. The method of claim 28, wherein the queuine or pharmaceutically acceptable salt or hydrate thereof is administered in a dosage regimen of from 0.01 mg/kg/d to 10 mg/kg/d.

30. The method of claim 29, wherein the queuine or pharmaceutically acceptable salt or hydrate thereof is administered in a dosage regimen of from 0.01 mg/kg/d to l mg/kg/d.

31. The method of claim 30, wherein the queuine or pharmaceutically acceptable salt or hydrate thereof is administered in a dosage regimen of from 0.01 mg/kg/d to 0.1 mg/kg/d.

32. The method of claim 27, wherein the queuine or pharmaceutically acceptable salt or hydrate thereof is administered orally, intradermally, intravenously, intramuscularly, or subcutaneously.

33. The method of claim 27, wherein the queuine or pharmaceutically acceptable salt or hydrate thereof is administered in combination with a compound selected from the group consisting of donepezil, galantamine, memantine, and rivastigmine.

34. The method of claim 27, wherein the queuine or pharmaceutically acceptable salt or hydrate thereof is administered in the form of a pharmaceutical composition comprising at least one pharmaceutically acceptable excipient or vehicle and queuine.

35. The method of claim 34, wherein the pharmaceutical composition further comprises a compound selected from the group consisting of donepezil, galantamine, memantine, and rivastigmine.

36. The method of claim 27, wherein the queuine or pharmaceutically acceptable salt or hydrate thereof is formulated as a dietary supplement.

37. The method of claim 36, wherein the dietary supplement comprises additional compounds selected from the group consisting of vitamins, minerals, fatty acids, amino acids, and antioxidants.
